(19) United States
(12) Patent Application Publication
(10) Pub. No.: US 2024/0344766 A1
(43) Pub. Date: Oct. 17, 2024

(54) INTELLIGENT REFRIGERATOR FOR THE STORAGE AND DISTRIBUTION OF PHARMACEUTICAL PREPARATIONS

(71) Applicant: CLINICAL SERVANT SPOLKA Z OGRANICZONA ODPOWIEDZIALNOSCIA, Lublin (PL)

(72) Inventors: Adam Kruszewski, Warsaw (PL); Jan Jakub Kruszewski, Warsaw (PL); Lukasz Stanislaw Olek, Jozefow (PL); Artur Brus, Lublin (PL)

(21) Appl. No.: 18/700,086

(22) PCT Filed: Oct. 10, 2022
(86) PCT No.: PCT/IB2022/059675

§ 371 Date: Apr. 10, 2024

(30) Foreign Application Priority Data
Oct. 12, 2021 (PL) 439187

(51) Int. Cl. F25D 29/00; A61J 7/00; G07F 17/00

(52) U.S. Cl. CPC F25D 29/008; A61J 7/0084; G07F 17/0092; A61J 2200/72; A61J 2200/74; F25D 2700/06

(57) ABSTRACT

Invention relates to a smart refrigerator for storing and distributing pharmaceutical products, consisting of a refrigeration device connected to a power source equipped with a casing and at least one shelf, at least one container for storing pharmaceutical products placed on at least one shelf, temperature sensors and a control unit with access to a computer network, characterised in that the container (5) for storing pharmaceutical products is essentially a cuboid equipped with a lid (3) coupled to an opening sensor (4) of the lid (3), and a weighing device integrated into a base of the container (5) and consisting of a weighing tray (1) and a strain gauge measuring system (2), the weighing device being connected to the control unit, consisting of a processor or programmable logic matrix connected to the relay device; the control unit comprises a memory and a set of algorithms managing the status of the refrigerator and an integrated notification and alarm system, while a control panel containing an interface and means for user verification connected to the lock mechanism and to the control unit is mounted in a refrigerator door.

The subject of the invention is a smart refrigerator for storage and distribution of pharmaceutical products.

Smart drug storage systems are known in the art. For example, document CN106395227 A, relates to a smart drug dispensing machine, which consists of a keel to support guide rail, a guide rail and a connection frame for a medicine container, guide rails and a guide rail driving mechanical arms for movement, a medication container, an item array for storing medicine storage baskets, universal medicine storage baskets, small medicine storage baskets, mechanical boxes, a mechanically driven arm to allow entry or extension of medicine storage baskets, a pneumatic mechanical arm to achieve the action to receive a medicine in the box, a medicine-receiving device, a medicine outlet channel, a drawer box with a weighing device, a medicine-positioning device, a smart medical frame, a special bi-verification system for the medicine cabinet, a control system for controlling the movement of the finishing device and a device for maintaining the heat to guarantee the regulation of the temperature in the device. The advantage of the device over known solutions is that all packaged medicines can be stored, special medicines are stored independently, medicines are taken in multiple modes, prescription only medication dispensing is achieved, the accuracy of medicine intake is automatically recalled, the number of medicines applied is automatically detected, automatic checking is achieved, and the temperature and humidity of medicine storage are regulated. The smart dosing machine is suitable for: hospital outpatient clinics, hospital pharmacies, commercial drug stores, small drug stores and more.

Document CN105395369A discloses a medicine reminder method, a terminal, a cartridge device and a dedicated system. Wherein, the disclosed method comprises obtaining medicine information for a user terminal; the terminal sends information to the means of the medicine cartridge, if the means of the cartridge for determining user information and said medicine dispensing information are the same, if not, the same medicine reminder message is generated, sending the medicine information to the terminal. The terminal then receives the medicine information; said terminal sends a reminder to the user concerning information on medicine use on the basis of the medicine. Wherein, the terminal, the kit, the device, the system accurately determine whether the user has taken the wrong medicine from the cartridge and automatically alerts the user when the user terminal is taking the wrong medicine to prevent errors.

Document PL415535 A1 discloses a refrigerator for medical purposes, consisting of a refrigeration device with shelves or drawers equipped with an automatic defrosting system and a casing of a refrigeration device with a transparent door with magnetic closure, characterised in that a transparent LCD screen is placed in the door, and a barcode reader is mounted on the casing of the refrigeration device, while at least two temperature meters are installed in the refrigeration device, and a door lock limiting switch is provided in the magnetic closure, the refrigerator being connected to the central unit. Connecting the refrigerator to a computer network enables the processing and collection of data regarding in particular: opening/closing the door (including scanning the employee card that opens the door—enabling identification of an employee); load insertion or removal (including data on the scanned products placed in the refrigerator, enabling the monitoring of the validity of the products); changing work settings; on-line monitoring.

Document PL295799 A1 discloses a method and an assembly for unambiguous association of medicines corresponding to a given prescribed treatment with a given patient. Wherein, said unit comprises a marking device, devices auxiliary for patient identification data, including a carousel of auxiliary devices and a transfer device connected to a power station from the control panel for reading patient identification data, a record of the prescribed treatment, reading identification data of a medicine to be dispensed and issuing a signal enabling the dispensing of this medication, wherein the control panel contains a reader of identification data marked on auxiliary devices attached to the patient and on containers with medicines, a reader of data associated with medicine packages, and a computer for recording prescribed treatments, and a device for preparing and filling the container with the prescribed medicine. The subject of the invention is also a method of unambiguous association of medicines corresponding to a given treatment, using the assembly described above.

Document PL224019 B1 discloses a smart drug box with a cuboid shape closed with walls constituting the top, bottom, rear and two side walls which consists of a body and at least one drawer, preferably fifteen drawers, wherein the body is divided into fifteen compartments of equal size by means of vertical and horizontal partitions, which contain removable drawers in the shape of cuboids open at the top. In addition, in each compartment of the body on the side opposite walls there are metallised conductive stripes connected to the control system, wherein the drawer on the outer side opposite walls has metallised conductive stripes which, when the drawer is placed in the body, are in surface contact with the corresponding metallised conductive stripes of the body. The drawer in the front wall has a handle for removing the drawer from the body. The drawer in the front wall has an optical indicator, preferably in the form of a diode. The control system is equipped with an acoustic signalling device as well as USB and Wi-Fi data links. The control system independently controls each of the diodes located in each of the fifteen drawers. The control system can activate the acoustic signalling device and detect the removal of each drawer by detecting the moment of disconnection of the diode power joints. The box has a wired or wireless communication interface for remote programming and control of medicine intake. The box has an option to program the time and day in which the medications from a given drawer are to be administered, so that they can be administered in other modes than the usual 3 times a day, while programming can be done using a smartphone or computer. The diodes in the remaining drawers and the acoustic signalling device will be used to signal that by mistake a wrong drawer was opened. The acoustic signalling device associated with each drawer can be programmed independently, which enables simultaneous administration of medicines in various modes, e.g. some medicines 3 times a day, some 4 times a day.

Document US2019086326 A1 discloses a device, system and method for medicine monitoring. The device for medicine monitoring includes a medicine storage container and a processing unit, wherein the medicine storage container is used for storing medications; and a processing module is used for analysing and confirming the amount and type of original medications in the medicine container and the amount and type of medicines removed from the medicine container, and the assessment whether the medications removed from the medicine container conform to the prescribed amount and type or not. Wherein, the device for medicine monitoring further comprises a medicine container, wherein the medicine container includes at least one medicine storage subunit, wherein each subunit has a through hole and is equipped with a provided laser device and a laser detection device, respectively, wherein the laser device is configured to emit laser radiation to be irradiated onto a drug surface in the subunit through the through hole, wherein the laser detection device is capable of receiving the laser light reflected back through the through hole and obtaining a spectrum of the medicine in the subunit; and each subunit is suitably equipped with a mass meter configured to detect the mass of the medicine within the subunit. The medicine monitoring device further comprises a memory circuit in which the tablet spectrum and weight for the plurality of medications has previously been stored.

Document WO2007084866 A2 discloses a refrigerator for storing pharmaceutical product containers, comprising: a cold storage compartment; a product identifier that reads a unique identifier produced by a pharmaceutical product container located in the cold storage compartment, wherein the product identifier has a read zone; a local database; an interface to a pharmaceutical product database comprising a set of details associated with the unique identifier; an event monitor coupled to a product identifier that will generate an added item event code in response to insertion of the pharmaceutical product container into the reading zone; and an event manager that responds as a result of receiving the added item event code. Wherein, if a set of detailed information related to the unique identifier already exists in the local database, the time of insertion of the pharmaceutical product container into the reading zone should be recorded. Otherwise, the dataset associated with the unique identifier is copied from the pharmaceutical database to the local database via the interface.

The aim of the invention is to provide a new smart refrigerator that improve the monitoring of the dispensing of pharmaceutical products.

The essence of the invention is a smart refrigerator for storing and distributing pharmaceutical products, consisting of a refrigeration device connected to a power source equipped with a casing and at least one shelf, at least one container for storing pharmaceutical products placed on at least one shelf, temperature sensors and a control unit with access to a computer network, characterised in that the container for storing pharmaceutical products is essentially a cuboid equipped with a lid coupled to an opening sensor of the lid, and a weighing device integrated into a base of the container and consisting of a weighing tray and a strain gauge measuring system, the weighing device being connected to the control unit, consisting of a processor or programmable logic matrix connected to the relay device; the control unit comprises a memory and a set of algorithms managing the status of the refrigerator and an integrated notification and alarm system as well as a relay device for transmitting data, while a control panel containing an interface and means for user verification connected to the lock mechanism and to the control unit is mounted in a refrigerator door.

Preferably, the lid is inclined with respect to the base of the container by an angle α ranging from 0 to 80°.

Preferably the lid is coupled with at least one LED indicator located on a wall of the container.

Preferably, the weighing device is built into the inner contour of the container above the base of the container, and the weighing tray is also a storage surface for pharmaceutical products.

Preferably, the refrigerator comprises at least one essentially cuboid auxiliary container equipped with a lid coupled with an opening detector or an opening detector and a LED indicator.

Preferably, the container is provided with an auxiliary lid.

Preferably, the refrigerator is equipped with a visual monitoring system in the form of at least one camera positioned above the at least one container.

Preferably, the control unit is networked with an external database containing data selected from the group comprising data on stored pharmaceutical products, patient registers, registers of people authorised to dispense stored pharmaceutical products, reports on the circulation of pharmaceutical products, codes assigned to pharmaceutical products, data from sensors, reference weights of individual products or a combination thereof.

Preferably, the user verification means comprises means selected from the group comprising a magnetic card reader, a retina scanner, a fingerprint reader or a combination thereof.

Preferably, through a network connection, the refrigerator is connected to a user equipment selected from the group comprising a computer, server, mobile device or a combination thereof.

The present invention provides the following advantages:
the invention ensures an increased safety of medicine dispensing, as it includes three integrated monitoring systems (i.e. weighing monitoring, compartment opening monitoring, LED monitoring);
the invention ensures an identification of the employee dispensing the medicine and the time of dispensing the medicine;
the invention provides authorised users with access to data on who dispensed a given medicine, what medicine was dispensed and for which patient;
the invention provides authorised users with real-time insight into the circulation of medicines placed in the refrigerator according to the invention, which allows for the determination whether the medicines were stored at the right temperature, how many medicines are in the entire refrigerator, and how many in individual containers, how many drugs have been dispensed from the entire device, and how many from a particular cartridge;
the invention provides access to reports and visualisations concerning the circulation of medicines, while the said reports and visualisations can be integrated with a smart, responsive website or with a dedicated application/software;
it eliminates the need to count dispensed medications and check the temperature logs of refrigerators used for clinical trials;
it provides a refrigerator temperature control;
it provides a protection of the refrigerator against an access by an unauthorised person;
it provides a real-time reporting of drug dispensing and refrigerator status to the device of the person responsible for the clinical trial;
it provides an automatic entry of the time of dispensing the medicine to the registry of the patient participating in a clinical trial in the database.

The invention is illustrated in the following embodiments and in the drawing in which

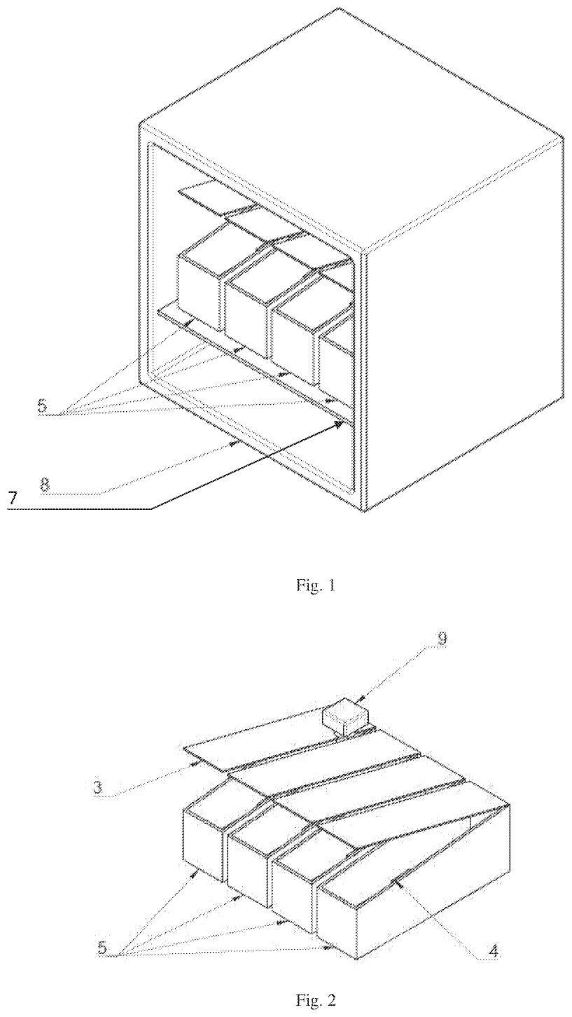

FIG. 1 shows a refrigerator according to the invention in a one-shelf version with four containers in variant I;

FIG. 2 shows the location of the visual monitoring system in a one-shelf refrigerator according to the invention with four containers in variant I;

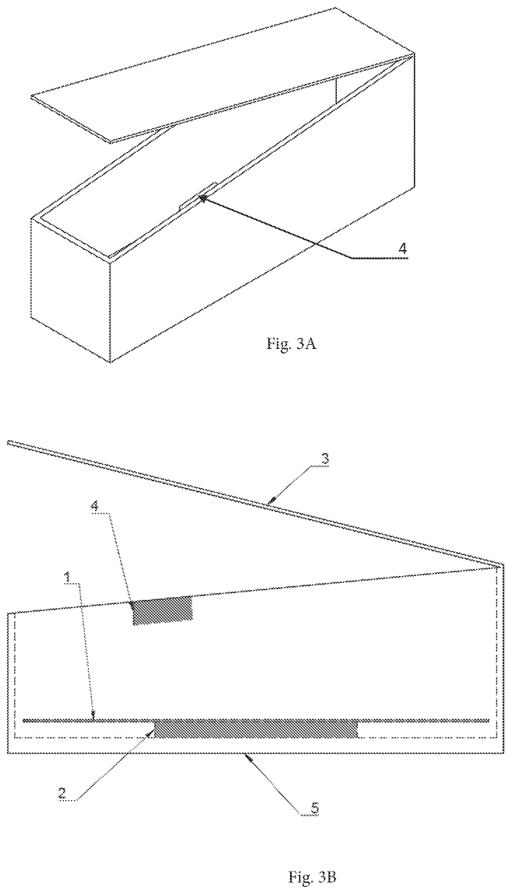

FIG. 3 shows a single container in variant I with a top opening lid A) in an isometric view, B) in a side view;

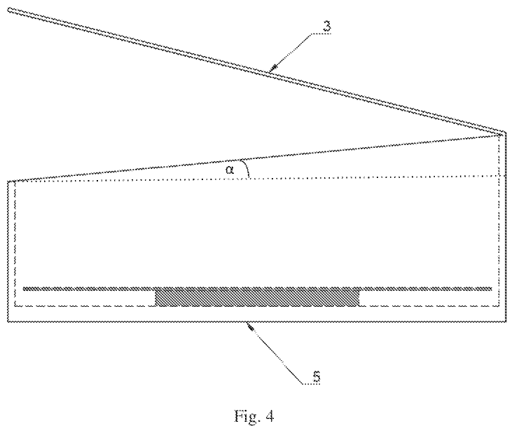

FIG. 4 shows a side view of a single container in variant I taking into account angle α;

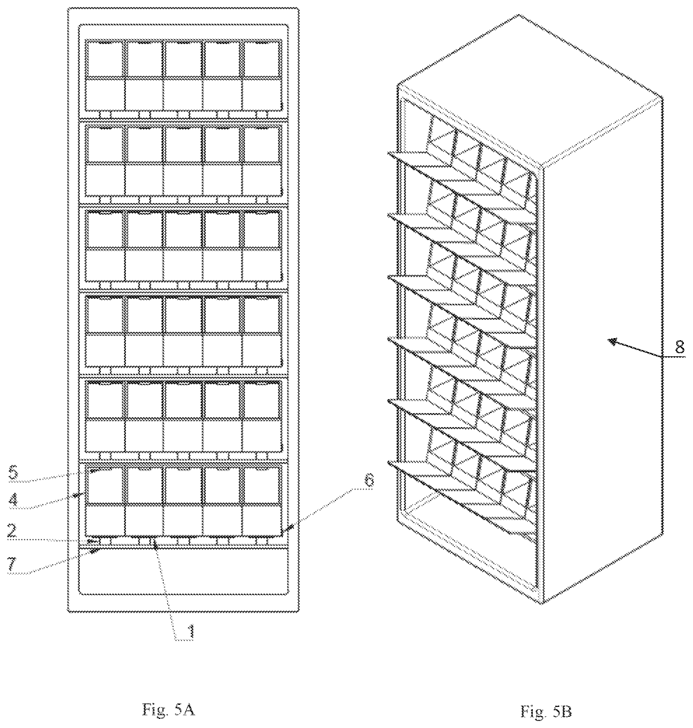

FIG. 5 shows a six-shelf refrigerator according to the invention with five containers in variant II on each shelf, where A) shows the refrigerator in a front view with the containers closed, B) shows the refrigerator in an isometric view with the containers open;

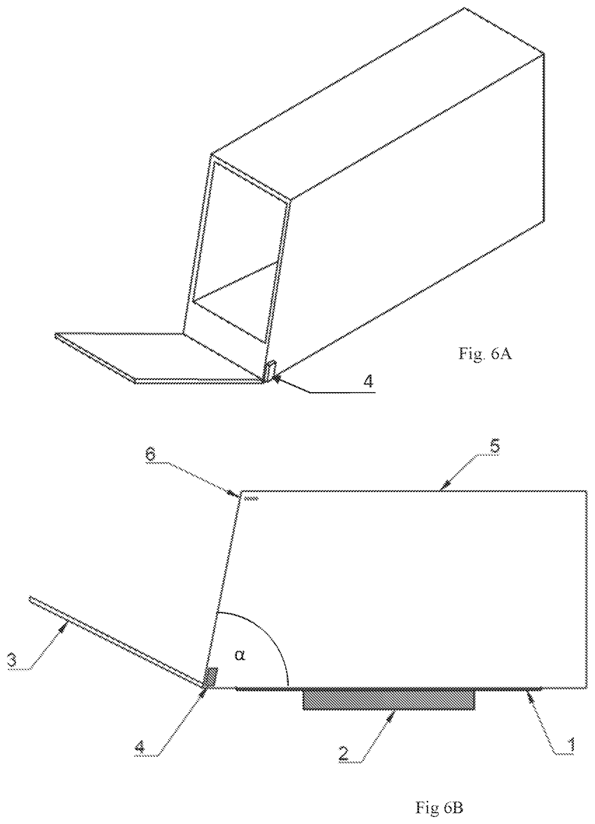

FIG. 6 shows a single container in variant II in an isometric view A), in a side view B);

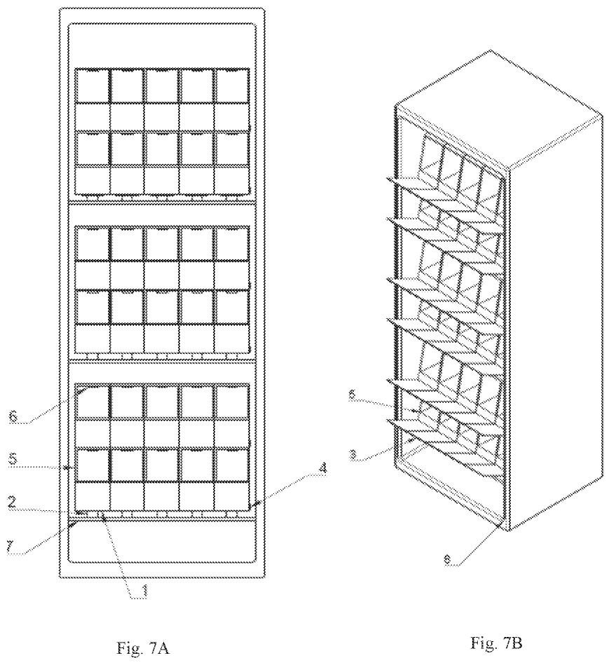

FIG. 7 schematically shows a refrigerator according to the invention in a three-shelf variant with containers according to variant II, on which one row of auxiliary containers is positioned, where A) shows a front view with closed containers and auxiliary containers, B) shows an isometric view with the open containers and auxiliary containers;

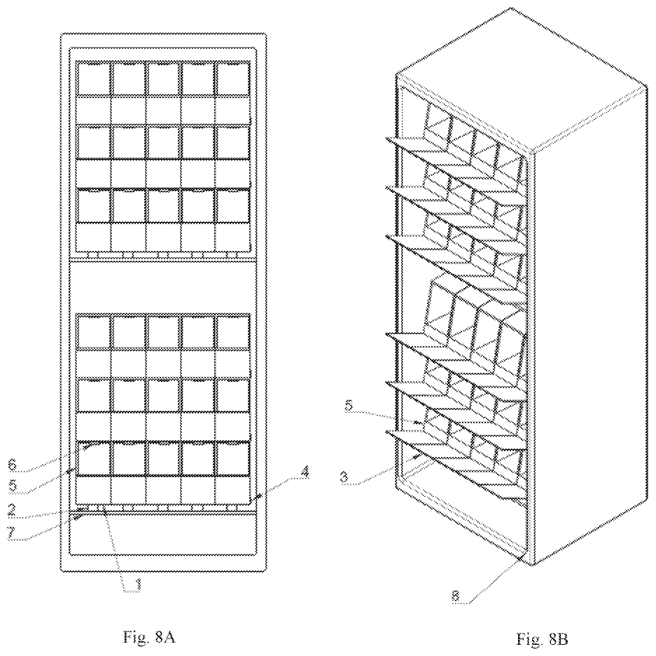

FIG. 8 schematically shows a refrigerator according to the invention in a two-shelf variant with containers according to variant II, on which two rows of auxiliary containers are positioned, where A) shows a front view with closed containers and auxiliary containers, B) shows an isometric view with the open containers and auxiliary containers;

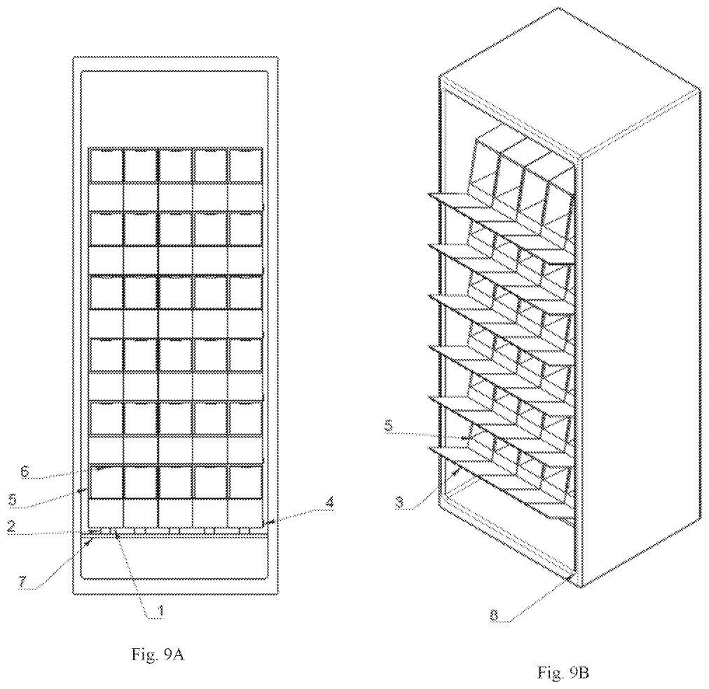

FIG. 9 schematically shows a refrigerator according to the invention in a one-shelf variant with containers according to variant II, on which five rows of auxiliary containers are positioned, where A) shows a front view with closed containers and auxiliary containers, B) shows an isometric view with the open containers and auxiliary containers;

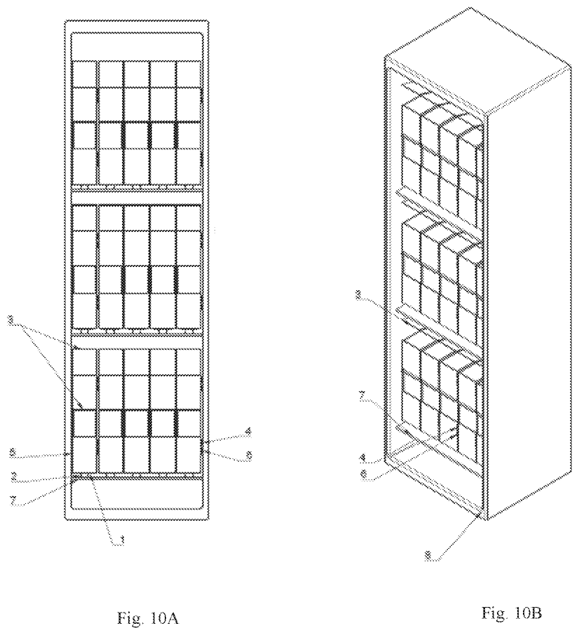

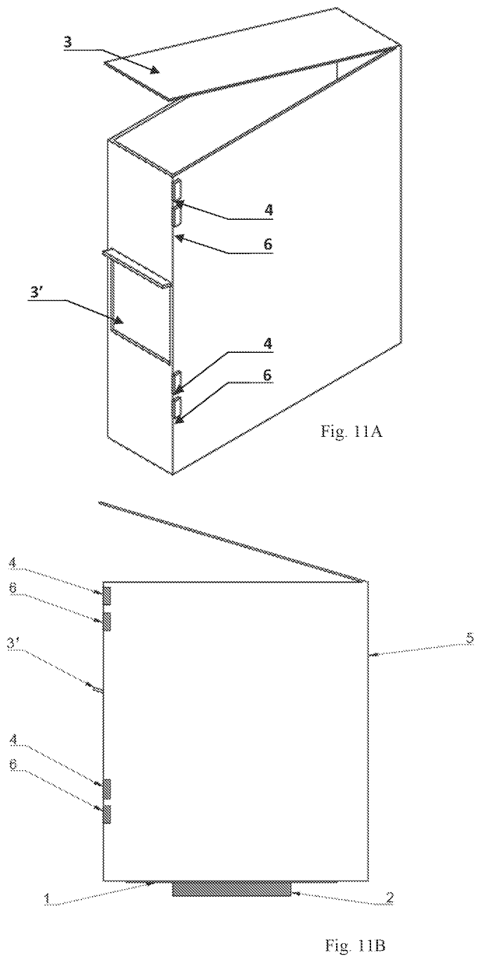

FIG. 10 schematically shows a refrigerator according to the invention in a three-shelf variant with containers according to variant III, where A) shows a front view, B) shows an isometric view;

FIG. 11 shows a single container in variant III in an isometric view A), in a side view B);

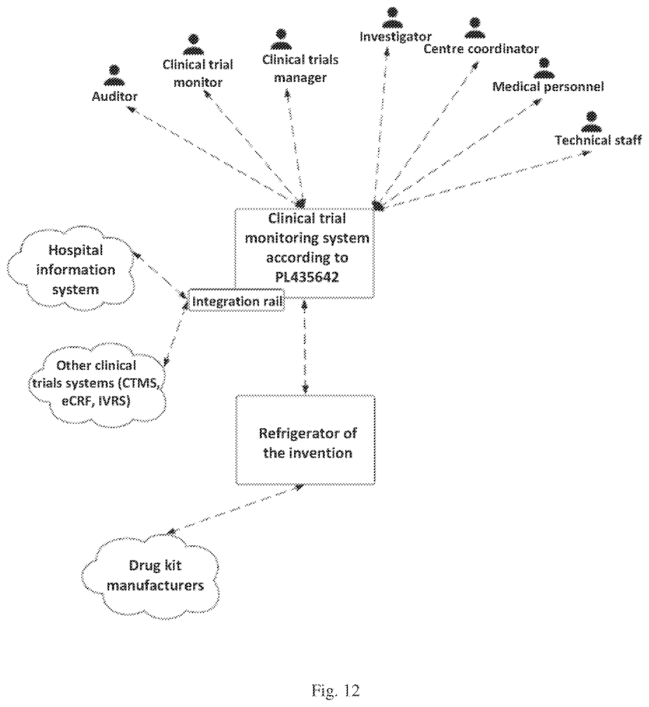

FIG. 12 shows a diagram for integrating a refrigerator with an external known clinical trial monitoring system.

### EXAMPLE 1

Smart refrigerator according to the invention is intended for the storage and distribution of pharmaceutical products. Wherein, within the meaning of the present invention, a pharmaceutical product may be a medicine, vaccine, buffer, disinfectant or other substance/mixture used in medicine or medical science. The said pharmaceutical products are stored in packages understood as each package containing a given product (e.g. a box, blister, bottle, vial, test tube, flacon, etc.).

In the present embodiment, the refrigerator according to the invention consists of a refrigeration device connected to a power source by means of a cable, wherein the refrigeration device is provided with a casing 8 and one shelf 7 on which four containers 5 for storing pharmaceutical products (FIG. 1) are placed. As indicated in FIG. 1-2, said containers 5 are arranged in series on shelf 7. Moreover, said refrigeration device comprises temperature sensors and a control unit with access to a computer network.

In this non-limiting embodiment, the interior of the refrigeration device and shelf 7 are made of stainless steel, while the exterior casing 8 of the refrigerator is made of aluminium. It is also possible to use other materials for the interior of the device, e.g. acid-resistant steel or aluminium. Shelves 7, in turn, can also be made of plastic (which meets the standards of materials suitable for use in a hospital) or glass. On the other hand, the casing 8 can also be made, for example, of powder coated steel.

In this embodiment, container 5 is of variant I (i.e. with a top opening lid) shown in FIG. 3. Wherein both walls and base of container 5 and its lid 3 are made of stainless steel. Wherein, it is also possible to use other materials, e.g. plastic.

As indicated in FIG. 3, in the present embodiment, the essentially cuboid container 5 is provided with a base, four side walls and a lid 3 closing said container 5 from above (by a hinge connection between the lid 3 and the rear wall of the container 5) and coupled to the opening sensor 4 of the lid 3 located on the inside of the side wall of the container 5 and connected (e.g. wirelessly) to the control unit, to which information regarding each opening of a given container 5 is transmitted.

Moreover, in this embodiment, a visual monitoring system 9 (FIG. 2) is mounted above one of the four containers 5 of the refrigerator according to the invention. Said monitoring system 9 is in the form of one camera placed above one of the containers 5 and it is directed towards the door of the refrigerator, providing additional supervision over opening the compartments, by registering the image of the person opening the refrigerator. The data from the camera is transmitted to a control unit containing the memory in which they are stored. In this embodiment, the visual monitoring system 9 consists of one camera, while the number of cameras may be greater depending on the dimensions of the refrigerator. In this embodiment, one camera is used because the refrigerator according to the invention comprises one short row of containers 5. One camera is enough for four containers. For longer rows, there may be many cameras in a row. Moreover, in the variants containing more than one shelf 7, each shelf 7 constituting a separate level with containers 5 is equipped with a camera or cameras.

In this non-limiting embodiment, the lid 3 is inclined with respect to the base of the container 5 by an angle α (FIG. 4) of 20°. On the other hand, according to the invention, other inclination of the lid 3 relative to the base of the container 5 may be used, ranging from 0 to 80°.

Furthermore, a weighing device, consisting of a weighing tray 1 and a strain gauge measuring system 2, is integrated in the base of the container 5, wherein said weighing device is connected to the control unit. Wherein, in this embodiment, the weighing device is built into the inner contour of the container 5 (indicated by the dashed line in FIG. 3B) above the base of the container 5, i.e. from the interior side of the container 5, so that the weighing tray 1 is also the surface on which the packages of stored pharmaceutical products are positioned.

The operation of the refrigerator is controlled by a control unit with an access to a computer network, which in this embodiment is mounted inside the casing 8. The control unit collects data from temperature sensors, opening sensors 4, a weighing device or a visual monitoring system 9, an interface on the refrigerator door. The control unit also manages the temperature settings, user authentication mechanism, lockout mechanism and alarm. Wherein, in this non-limiting embodiment, the control unit is a processor (but a programmable logic matrix or other device allowing for arithmetic and logical operation may also be used) connected to a relay device for transmitting data over wired links. However, wireless links (e.g. Ethernet, USB, Bluetooth, WiFi, cellular network) or a combination of wired and wireless links can also be used. The processor includes memory, a set of algorithms that manage the status of the refrigerator, and an integrated notification and alarm system. The memory can store e.g. device operation programmes, temperature range above/below which the alarm is triggered, correction factors for temperature sensors and weighing devices, data of people managing the alarm and notification mode, model data for biometric verification, codes of pharmaceutical products, standard weights of individual products, etc. The control unit can also send unprocessed data directly from sensors installed inside the refrigerator to an external server where such processing will be performed. In the event of lack of communication with the server (e.g. because of a network failure or failure to connect the refrigerator according to the invention to the network), the control unit can save its internal states, events and sensor data in the built-in memory, and send historical data once the communication with the server is restored.

In this embodiment, the control unit is provided with several communication channels, in the event of no communication with the server via local network access, the management algorithms may attempt to connect via another communication channel, e.g. via a cellular network. In this embodiment, the control unit comprises a real-time clock from which it obtains the current date and time so that in the absence of a network connection it is possible to save these parameters until the change of a status or event that has occurred.

In addition, in this embodiment, the control unit is equipped with emergency power supply systems (e.g. with an emergency battery built into the base of the casing 8), enabling the continuation of its work in the absence of external power, which allows the measurement devices to continue working and to send a power failure message to the operating staff. The refrigeration device of the refrigerator according to the invention can also be equipped with emergency power supply systems, which will allow for a suitable temperature inside for the time needed for the reaction of the operating staff.

In this embodiment, the control unit is provided with an additional interface for connecting external devices e.g. a barcode scanner, NFC tag reader (i.e. short range communication).

Furthermore, the refrigerator according to the invention can be combined with known clinical trial management systems. In this embodiment, the control unit is networked with an external database (e.g. located on a hospital server) containing in particular data on the stored pharmaceutical products (e.g. batch number, doses, type of packaging, number of doses in the package), patient records (e.g. including medical record, prescribed drugs, data of the attending physician), registers of people authorised to issue stored pharmaceutical products, reports on the circulation of pharmaceutical products, codes assigned to pharmaceutical products, etc.

Algorithms managing the status of the refrigerator enable programming of the device (e.g. operating mode), as well as processing of data obtained from the opening sensors 4, the weighing device, the visual monitoring system 9, the temperature sensors or the interface on the refrigerator door. Therein, the communication of the refrigerator control unit with the database server may be unidirectional or bidirectional. On the other hand, data transmission by the managing system to the server can be triggered by various events, e.g. opening of the refrigerator door, temperature increase/decrease, change in the weighing device readout. Reading the current parameters and the status of the refrigerator sensors can also be triggered by sending an appropriate command from the server. Wherein, each event has its own time marker, downloaded from the NTP time server (Network Time Protocol), or in the case of no connection with the server, time is downloaded from the real-time clock integrated into the control unit. The real-time system can be synchronised with NTP when it is available.

The control unit contains built-in algorithms to remotely update the firmware. In this embodiment, said control unit firmware update is signed with a digital certificate ensuring the authenticity of the update file. Each control unit has its own unique hardware identification number by which it is recognised by the management server as belonging to a specific instance of the refrigerator according to the invention.

The reading of the values from the built-in sensors can be controlled by the built-in timer (measurement at a specified time interval), an event, e.g. opening the refrigerator door, an appropriate message from the management server. Data from sensors, visual monitoring system 9, interface on the refrigerator door, or additional devices connected to the control unit (e.g. barcode scanner) can be stored in the memory of the control unit, in the memory of the management server or using a hybrid mode with synchronisation between them at a specified time interval. All data can be processed by the systems of the control unit or by the management server. The control unit may communicate with the database server via the local computer network at the installation site or via other channels, e.g. a cellular network.

The control unit can use the local network to communicate with the server if both devices are located in the same building or via the Internet if the management server is located elsewhere. The control unit may have the function of automatic testing of its correct operation, as well as of connected sensors and devices.

The weighing device (i.e. a weighing scale) is connected (e.g. wirelessly) to a control unit which on the basis of weight changes determines whether, when and how many pharmaceutical products have been dispensed, which allows the contents of the containers 5 to be monitored in real time. Data on the intake of a given amount of the product by a given physician is sent to that physician's record in the medical personnel database. In this embodiment, the weighing device is an electronic scale with an accuracy of 0.5 grams, and a weighing capacity of the scale is 1 kg. This accuracy enables the control unit to decide whether or not the amount of a given drug increased or decreased. However, more accurate scales can be used.

The door of the refrigerator according to the invention is fitted with a locking mechanism and a control panel containing a user interface displaying the current date and allowing for the programming and changing of the operating settings of the refrigerator. In particular, it allows for the programming of the date and time, the set temperature of the refrigerator, the range of safe temperature operation, the temperature range below and/or above which an alarm and a notification for the user about the failure are generated via the control unit notification system. Therein, the refrigerator can be programmed remotely from the level of the device (e.g. a smartphone, computer) of an authorised user via a dedicated application.

The control unit notification system informs the authorised user (e.g. via SMS, e-mail, recorded telephone message) of any problems related to the storage of medicines in the device according to the invention (e.g. due to a not properly closed door or inappropriate temperature). Wherein, depending on the settings selected by the user, said notifications may also relate to each opening of the refrigerator door, each dose of a selected pharmaceutical product which was dispensed, or information about the supply status of the refrigerator (e.g. by generating a notification each time the container 5 is completely empty).

In this embodiment, the control panel is equipped with an LCD display on which the user can verify the temperature log of the refrigerator operation, check the history of opening/closing the door, and the history of medicine dispensing. Moreover, the control panel is provided with means for user verification connected to a lock mechanism and to a control unit, which ensure the identification of the person taking pharmaceutical products in real time.

In this embodiment, the verification means comprise a magnetic card reader and a fingerprint reader. Other security features can also be used, e.g. a retinal scanner, access code, key lock, etc. The number of security measures used is unrestricted, while double verification itself increases security, and the use of individual user features, such as a fingerprint, eliminates the risk of unauthorised access as a result of e.g. theft of a card or access code.

The high-tech smart refrigerator according to the invention can be used, for example, in a hospital as a management and monitoring system for dispensing medicines, as a device supporting and monitoring medicine dispensing in clinical trials, and as a safe storage system for experimental drugs.

### EXAMPLE 2

Refrigerator as in Example 1, except that it is equipped with thirty containers 5 arranged in series on six shelves 7, and the visual monitoring system 9 consists of twelve cameras placed above the containers 5 in an arrangement of two cameras for each shelf 7. As indicated in FIG. 5, on each of the six shelves 7 five containers 5 in variant II (i.e. with a front opening lid) are arranged in series. Wherein, each of the containers 5 is large enough to accommodate a human hand.

The structure of the container 5 in variant II is schematically shown in FIG. 6. As indicated in FIG. 6, the lid 3 opens from the front, i.e. from the portion of the container 5 located frontally with respect to the user opening the refrigerator (as schematically shown in FIGS. 5A and 5B). Moreover, the lid 3 is coupled to the opening sensor 4 of the lid 3 located at the base on the outside of the side wall of the container 5 and connected (e.g. wirelessly) to the control unit, to which information regarding each opening of a given container 5 is transmitted. Each opening of the container 5 is additionally indicated by an LED indicator 6 located on the outer side of the side wall at its upper edge. The light signal generated after opening a given container 5 is clearly visible from the user's perspective.

In this embodiment, the mobility of the lid 3 is based on a hinge mounted between the lid and the base of the container 5, wherein the walls of the container 5 are curved at the top, which facilitates the closing of the container 5 and reduces the risk of the contents of the container 5 “spilling out”. Moreover, in this non-limiting embodiment, the lid 3, when closed, is inclined with respect to the base of the container 5 by an angle α (FIG. 6B) of 80°. On the other hand, a different inclination of the lid 3 relative to the base of the container 5 can be used. Furthermore, a weighing device 5, consisting of a weighing tray 1 and a strain gauge measuring system 2 is integrated in the base of the container, the weighing device being connected to the control unit. Wherein, as indicated in FIG. 6 the weighing device is integrated into the outer part of the base of the container.

### EXAMPLE 3

Refrigerator as in Example 1, except that it is equipped with three shelves 7, wherein on each of the shelves 7 five containers 5 according to variant II are arranged in series, and on each of them one auxiliary container 5′ (FIG. 7) is placed. On the other hand, the visual monitoring system 9 consists of six cameras located above the auxiliary containers 5′ in a system of two cameras for each shelf 7, wherein the cameras are directed towards the user, which allows for monitoring both the status of each shelf separately and the people using the refrigerator.

The structure of the container 5 is analogous to that in Example 2, except that the LED indicator 6 is positioned in front of the container 5 above the edge of the lid 3 in such a way that when the container 5 is opened, the LED indicator is perceived centrally by the user (FIG. 7A). On the other hand, essentially cuboid auxiliary containers 5′ are equipped with a lid 3 coupled with an opening sensor and a LED indicator 6. Their dimensions and the way of opening the lid 3 match the containers 5 on which they are placed. In such variant of the refrigerator according to the invention, the weighing device integrated into the base of the container 5 is responsible for monitoring the weight changes of the entire column formed of the container 5 and the auxiliary container 5′ placed thereon. On the other hand, the very differentiation from which of the column elements the medicine dose was removed is performed by the control unit on the basis of data from the appropriate reader 4 and supported by the analysis of the image from the camera of the visual monitoring system 9.

### EXAMPLE 4

Refrigerator as in Example 3, except that it is equipped with two shelves 7, wherein on each of the shelves 7 five containers 5 according to variant II are arranged in series, and on each of them two auxiliary containers 5′ are placed on top of each other, forming a column of three containers (FIG. 8). On the other hand, the visual monitoring system 9 consists of four cameras placed above the topmost auxiliary containers 5′ in the arrangement of two cameras for each shelf 7.

### EXAMPLE 5

Refrigerator as in Example 3, except that it is equipped with one shelf 7, on which five containers 5 according to variant II are arranged in series, and on each of them five auxiliary containers 5′ are stacked consecutively to form a column of six containers (FIG. 9). On the other hand, the visual monitoring system 9 consists of two cameras positioned above the second and fourth top auxiliary containers 5′.

### EXAMPLE 6

Refrigerator as in Example 1, except that it is equipped with fifteen containers 5 arranged in series on three shelves 7, and the visual monitoring system 9 consists of six cameras placed above the containers 5 in an arrangement of two cameras for each shelf 7. As indicated in FIG. 10, on each of the six shelves 7 five containers 5 in variant III (i.e. in the enlarged variant with the auxiliary lid 3′) are arranged in series.

The structure of the container 5 in variant III is schematically shown in FIG. 11. The container 5 is provided with a base, four side walls and a lid 3 closing said container 5 from above (by a hinge connection between the lid 3 and the rear wall of the container 5) and coupled to the opening sensor 4 of the lid 3 located on the outside of the side wall of the container 5 and connected to the control unit, to which information regarding each opening of a given container 5 is transmitted. Below the aforementioned sensor 4 a LED indicator 6 is placed. Furthermore, a weighing device 5, consisting of a weighing tray 1 and a strain gauge measuring system 2, is integrated in the base of the container, the weighing device being connected to the control unit.

As indicated in FIG. 11, the lid 3 is parallel to the base of the container 5 (angle α=0°). On the other hand, on the front wall (i.e. the wall facing the user opening the refrigerator door), halfway up its height, there is an auxiliary lid 3′ that opens to the front (i.e. towards the front of the user opening the refrigerator door).

Wherein, said auxiliary lid 3′ is coupled to the second opening sensor 4 and the second LED indicator 6 positioned on the outer side of the side wall of the container 5 at the height of the auxiliary lid 3′. The auxiliary cover 3′ cooperates with the opening sensor 4 and provides information about the interaction of the user with the contents of the inner chamber of the container 5.

### EXAMPLE 7

Refrigerator as in example 6, except that the flap of the container 5 is equipped with an additional handle for easy opening. However, instead of an additional handle, a flap with an hole for easy opening can be applied.

### EXAMPLE 8

Refrigerator as in Example 6, except that it is equipped with twenty five containers 5 arranged in series on five shelves 7, and the visual monitoring system 9 consists of ten cameras placed above the containers 5 in an arrangement of two cameras for each shelf 7.

Wherein, in this non-limiting embodiment, the container 5 is characterised by the following dimensions:
height up to 19.6 cm;
length up to 10.6 cm;
width up to 12 cm.

### EXAMPLE 9

The refrigerator according to the invention can be combined with known clinical trial management systems. In this embodiment, the refrigerator according to the invention is connected to the system for automatic monitoring of clinical trials known from the application PLA35642.

For this purpose, the control unit of the refrigerator according to the invention is networked with an external information system according to the application PL435642 (hereinafter referred to as External System), which will constitute the control and communication function between the investigator and the refrigerator according to the invention. In particular, the External System according to the application PL435642 contains a logical representation of the principles of a given clinical trial, the source of which is the clinical trial protocol. After the start of the trial, information from randomisation of patients is received (it can originate from external systems, e.g. IVRS or be directly generated by the External System according to the application PL435642), which allows for the planning of the visit schedule and the necessary sets of medicines divided by individual participants of the trial. Based on the above information, orders for medicine packages dedicated to individual participants, needed at a specific point in time, which will be fulfilled by medicine manufacturers, are generated. In addition, the refrigerator according to the invention will generate, for the External System according to the application PL435642, control information concerning loading the container 5, retrieving the medicine packet as well as about other events necessary to manage and monitor the status of the refrigerator (e.g. functional status of the refrigerator, date and time of the medicine intake, data of staff collecting the drug, patient data, medicine data such as type of product, dose or batch number).

Communication with the user will take place via the user interface programmed in applications served on devices containing a processor (e.g. a laptop, tablet, smartphone). On the other hand, the communication between the system according to the application PL435642 and the control unit of the refrigerator according to the invention will be achieved via a dedicated integration layer of the External System.

The starting point for activating the connection between the refrigerator and the External System is to run a trial for that patient. Then, the External System, based on the information contained in the clinical trial protocol, will generate information about what medicines and when should be administered for the indicated patient. Based on this information, reports for manufacturers of medicine packages will be generated, and medicine packages will be prepared and delivered to the centre in a manner allowing for their identification (e.g. encoded with QR codes originating from the External System). These packets will then be placed in the smart container, i.e. in the containers 5 of the refrigerator according to the invention, where they will be awaiting their removal by the clinical trial staff at the centre at the appointed time and for the relevant patient. Information (schedule of visits) will be available via the application in the External System, while additional communication with the user will take place via the display of the smart refrigerator according to the invention. The External System communicates on an ongoing basis with the refrigerator according to the invention, transmitting one-way information about the packages of medicines that should be placed in the refrigerator. On the other side, the refrigerator according to the invention informs the External System about the insertion and/or removal of the package. Based on this information, appropriate algorithms are run both in the External System and in the control unit of the refrigerator according to the invention.

For example, after selecting the ongoing visit of a study participant in the External System application, the smart refrigerator according to the invention will highlight the relevant drawer containing the medicine package for that participant assigned to a given visit. During the administration of the medicines, it is finally verified whether the collected set is correct, e.g. by QR codes (or other markings allowing for unambiguous selection, e.g. a bar code), which constitute a code identifying the set that has previously been assigned to it and associated with the visit of a given patient, by the External System.

Additionally, the External System may be integrated with a hospital system and/or other systems as schematically shown in FIG. 12.

1. A smart refrigerator for storing and distributing pharmaceutical products, consisting of a refrigeration device connected to a power source equipped with a casing and at least one shelf, at least one container for storing pharmaceutical products placed on at least one shelf, temperature sensors and a control unit with access to a computer network, characterised in that the container (5) for storing pharmaceutical products is essentially a cuboid equipped with a lid (3) coupled to an opening sensor (4) of the lid (3), and a weighing device integrated into a base of the container (5) and consisting of a weighing tray (1) and a strain gauge measuring system (2), the weighing device being connected to the control unit, consisting of a processor or programmable logic matrix connected to the relay device; the control unit comprises a memory and a set of algorithms managing the status of the refrigerator and an integrated notification and alarm system, while a control panel containing an interface and means for user verification connected to the lock mechanism and to the control unit is mounted in a refrigerator door.

2. The refrigerator according to claim 1, characterised in that the lid (3) is inclined with respect to the base of the container (5) by an angle α ranging from 0 to 80°.

3. The refrigerator according to claim 1 or 2, characterised in that the lid (3) is coupled with at least one LED indicator (6) located on a wall of the container (5).

4. The refrigerator according to any of the preceding claims from 1 to 3, characterised in that the weighing device is built into the inner contour of the container (5) above the base of the container (5), and the weighing tray (1) is also a storage surface for pharmaceutical products.

5. The refrigerator according to any of the preceding claims from 1 to 4, characterised in that it comprises at least one essentially cuboid auxiliary container (5′) equipped with a lid (3) coupled with an opening detector (4) or an opening detector (4) and a LED indicator (6).

6. The refrigerator according to any of the preceding claims from 1 to 4, characterised in that the container (5) is provided with an auxiliary lid (3′).

7. The refrigerator according to any of the preceding claims from 1 to 6, characterised in that it is equipped with a visual monitoring system (9) in the form of at least one camera positioned above the at least one container (5).

8. The refrigerator according to any of the preceding claims from 1 to 7, characterised in that the control unit is networked with an external database containing data selected from the group comprising data on stored pharmaceutical products, patient registers, registers of people authorised to dispense stored pharmaceutical products, reports on the circulation of pharmaceutical products, codes assigned to pharmaceutical products, data from sensors, reference weights of individual products or a combination thereof.

9. The refrigerator according to any of the preceding claims from 1 to 8, characterised in that, the user verification means comprises means selected from the group comprising a magnetic card reader, a retina scanner, a fingerprint reader or a combination thereof.

10. Refrigerator according to any of the preceding claims from 1 to 9, characterised in that the refrigerator is connected to a user equipment selected from the group comprising a computer, server, mobile device or a combination thereof.
